(19) United States
(12) Patent Application Publication
(10) Pub. No.: US 2025/0295903 A1
(43) Pub. Date: Sep. 25, 2025

(54) MICRONEEDLE DRUG DELIVERY DEVICES AND METHODS FOR USING THE SAME

(71) Applicant: Tycho J. Speaker, Santa Cruz, CA (US)

(72) Inventor: Tycho J. Speaker, Santa Cruz, CA (US)

(21) Appl. No.: 19/230,795

(22) Filed: Jun. 6, 2025

Related U.S. Application Data
(63) Continuation-in-part of application No. PCT/US2024/059519, filed on Dec. 11, 2024.
(60) Provisional application No. 63/608,454, filed on Dec. 11, 2023.

(51) Int. Cl. A61M 37/00

(52) U.S. Cl. CPC A61M 37/0015; A61M 2037/0023

(57) ABSTRACT

A drug delivery device has a post structure having a base and at least one post protruding from the base. A deformable tray is movably coupled to the post structure and defines at least one cavity extending from an upper surface of the tray toward a lower surface of the tray. The at least one cavity tapers to a point at or near the lower surface of the tray. At least one microneedle is positioned within the at least one cavity. At least one reservoir is positioned directly above and axially aligned with the at least one cavity. The reservoir is configured to contain a therapeutic agent. An adhesive surface is positioned on the lower side of the deformable tray. The adhesive surface is configured to adhere to the skin and optionally remain on the skin when the microneedle device is withdrawn.

This application a continuation-in-part (CIP) of PCT Application Ser. No. PCT/US2024/059519, filed 11 Dec. 2024, which claims the benefit of priority of U.S. Provisional Application No. 63/608,454, entitled MICRONEEDLE DRUG DELIVERY DEVICES AND METHODS FOR USING THE SAME, filed on 11 Dec. 2023, the contents of all of which are incorporated herein by reference in their entireties for all purposes.

### FIELD OF THE INVENTION

This invention relates generally to the field of intracutaneous drug delivery and to related products and processes for making and using products for this purpose. The invention further relates to the manufacture of microneedle products and systems including forming, loading, and depositing microneedle structures into biological tissues, and particularly into the skin. The invention further relates to the field of controlled or sustained release of agents from implanted microneedles. The invention further relates to the field of vaccination, and particularly cutaneous vaccination.

The invention further relates to adhesive-based delivery systems that can adhere to the skin upon application, thereby enhancing drug delivery outcomes via occlusion, moisture retention, or sustained transdermal diffusion. The invention further relates to controlled or sustained release systems for drugs, vaccines, or other therapeutic agents and to methods for manufacturing and deploying these systems using existing automated aseptic manufacturing lines.

### BACKGROUND OF THE INVENTION

The use of microneedles for intracutaneous drug delivery and vaccination is well established in commerce and in the literature, and many varieties of microneedle structures, compositions, and devices have been developed to deliver active agents to the skin and other tissues. Microneedles penetrate through the barrier posed by the exterior of the skin to deliver their payload to the live layers of skin and to the underlying tissues.

The term “microneedle” refers generally to needles of very small dimensions relative to common syringe-and-needle drug delivery, and microneedle technology is characterized by needle lengths appropriate to intradermal rather than intramuscular delivery. Generally, microneedles refer to roughly conical or pyramidal structures, comprising a wider base supporting a structure that tapers to a sharp tip. The tip end must be sharp enough to penetrate skin when a force is applied, having a cross-sectional diameter of typically less than 100 microns. The force required to insert the needle is influenced by the degree to which the microneedle structure widens along its length, conveniently referred to as its taper or “aspect ratio”, the ratio of width to height, e.g. a structure that widens from a sharp tip to a 200 micron base over a 1000 micron length has an aspect ratio of 1000/200=5. Typically, microneedles have an aspect ratio of greater than about 3, as decreasing aspect ratio limits penetration into skin, and generally the larger the aspect ratio the less force is required to insert.

Microneedles can be broadly divided into several categories, such as hollow vs solid microneedles. Hollow microneedles are designed for the passage of fluids, e.g. for either delivery or extraction. By contrast, solid, dissolvable microneedles are commonly provided as microneedle array patches (MAPs) in which multiple microneedle structures are formed attached to a common backing layer as part of a larger patch. Such patches provide a range of benefits including ease of handling of the tiny microneedle structures and the benefit of orienting the tips perpendicular to a backing layer, which can be conveniently pressed or otherwise applied to the skin as a monolithic structure comprising tens, or even thousands of microneedles. It is common to apply many microneedles to deliver a single dose, as each microneedle is only capable of delivering a very small amount of material to the tissue target. For example, a typical conical or pyramidal polymer microneedle of 1 mm height and an aspect ratio of 5 may be calculated (V=(nr2h)/3) to have a volume in the range of 50 nanoliters and may be expected to weigh on the order of 50-100 nanograms. Therefore a 100-needle MAP in which the needle structures are composed entirely of the drug substance might contain at most 100*0.1 mg, or a 10 mg drug payload. In practice, it is not always reasonable to form a microneedle exclusively from an active ingredient, reducing this maximal payload. If the microneedle composition does not include an active ingredient, it is frequently coated, then dried on the exterior surface of the needles, which further limits payload volumes in such devices. Yet further limiting the payload delivery capacity of MAPS is the fact that the microneedle projections typically do not penetrate the skin to their full height, due to local stretching and indentation of the skin at the point of contact. When the skin indents at the point of contact, the skin between the microneedles is elevated with respect to the tips, and makes contact with the contiguous backing layer before needles are completely inserted, effectively limiting the depth to which the needles can penetrate.

Existing vaccine production for global vaccines is enormous, at 16 billion doses of 47 different vaccines in 2022, according to the World Health Organization. This well-developed production capacity is dominated by high-speed automated aseptic filling lines engineered to fill, lyophilize, and cap glass vials, which are reconstituted for use. Currently there is no viable technology to bridge the manufacturing methods used for such lyophilized vial products and the world of microneedle-based vaccines. The gap between real-world mass vaccine production and the most advanced microneedle loading technology today remains daunting.

Thus, an acute need exists for a microneedle technology that can be loaded with vaccine on an existing automated filling line with minimal adaptation or modification of standard equipment or procedures. There also exists a need for a versatile microneedle-based delivery system which accommodates multiple types of formulations, including non-lyophilized materials and provides controlled or sustained release of therapeutic agents, all in a singular device suitable for mass-production using existing commercial aseptic filling lines.

### SUMMARY OF THE INVENTION

Aspects of the invention provide freestanding microneedle tips supported by a deformable support and connected reservoir such that the tips may be loaded by dispensing a volume of liquid into the reservoir and drying it upon the tips by lyophilization or other means. Further, aspects of the invention provide a means of delivering the loaded, dried tips by means of posts that drive the tips through the deformable support and into underlying skin or other tissue. Still further, other aspects of the invention provide dramatically enhanced penetration of the needle tips into skin via the support's engagement of the skin providing a localized stretching effect, combined with the post's travel beyond the plane of the support. Other aspects of the invention provide a vial-shaped enclosure containing the supported tips and post structures, suitable for automated handling in an aseptic filling line. Aspects of the invention further provide a vial-shaped enclosure incorporating an actuator that drives the loaded tips through the support and into underlying skin or other tissue. In another embodiment the invention comprises a sterile barrier, single-use, non-reconstituted, self-contained device for vaccine administration.

Aspects of the invention also provide free-standing, pointed microneedle structures composed of bioresorbable materials, engaged in a fixed orientation by a deformable tray or support, and associated post structures arranged such that these can be moved relative to the microneedle structures, driving them through deformable support. The posts, microneedles, and support are configured such that when the support is placed against a surface, for example human skin, actuating the posts will drive the microneedles into the underlying surface. In another aspect, the invention provides a set of reservoir chambers of similar cross-section to the microneedles and posts, and in a matching arrangement such that the reservoirs are positioned directly over the microneedles, and between the microneedles and the posts. In this configuration, when the invention is deployed, the posts transit first through the reservoirs, initially compressing any contents (typically a dry composition) against the back of the microneedles, and subsequently driving the microneedles along with the reservoir contents into underlying skin. Still further aspects of the invention include engagement of the skin in contact with the deformable or elastomeric support such that local deformation is minimized, which in conjunction with the absence of a backing layer and the capacity to drive the posts substantially through the support structure, provides a dramatic enhancement of needle penetration depth into the underlying skin. Another aspect of the invention provides an actuator mechanism that permits a triggered deployment of the posts and safeguards against accidental deployment. Another aspect provides an enclosure that permits filling of the reservoir structures in the context of existing aseptic automated filling lines, as well as in situ lyophilization of the reservoir contents, and serves as a sterile enclosure until deployed in use. In another aspect the invention provides a device comprising deployable posts and needle structures that will accept reservoir structures preloaded with different compositions as a rapidly configurable delivery system for different active agents. In another embodiment, the invention comprises a compact, sterile barrier, single-use, non-reconstituted, self-contained device for intradermal administration of materials into the skin, such as therapeutic agents or vaccines.

In accordance with one aspect of the present invention, a drug delivery device includes a post structure and a deformable tray movably coupled to the post structure. The post structure has a base and at least one post protruding from the base. The deformable tray has an upper surface facing the post structure. The deformable tray defines at least one cavity extending from the upper surface of the tray toward a lower surface of the tray. The at least one cavity tapering to a point at or near the lower surface of the tray. The at least one post is aligned with the at least one cavity such that movement of the post structure relative to the deformable tray moves the at least one post toward the upper surface of the tray and into the at least one cavity so as to exert a force on a microneedle in the at least one cavity sufficient to drive the microneedle through the lower surface of the tray.

In some aspects of the present invention, the drug delivery device further includes at least one reservoir positioned directly above and axially aligned with the at least one cavity. The at least one post is further aligned with the at least one reservoir such that movement of the post structure relative to the deformable tray moves the at least one post through the at least one cylindrical reservoir so as to exert a force on contents of the at least one reservoir sufficient to drive the contents through the lower surface of the tray.

In some aspects of the present invention, the at least one reservoir has a diameter corresponding to a diameter of an upper surface of the cavity. Additionally or optionally, the contents of the at least one reservoir comprise a dried and/or solid drug payload positioned within the at least one reservoir.

In certain aspects of the present invention, the at least one reservoir is defined by a rigid plate positioned above the deformable tray. Additionally or optionally, the rigid plate is positioned in contact with the deformable tray.

In some aspects of the present invention, the post structure comprises a cartridge, and the base forms at least part of an inner surface of the cartridge. Additionally or optionally, the cartridge has a cylindrical cross-sectional shape and an upper end of the cartridge has a size corresponding to a standardized vaccine vial. In still other aspects of the present invention, the drug delivery device further includes a fluid pathway extending from the upper end of the cartridge through an interior space of the cartridge to the at least one reservoir. Additionally or optionally, walls of the fluid pathway comprise a hydrophobic coating or material.

In some aspects of the present invention, the drug delivery device further includes a sleeve movably coupled to the post structure and the sleeve is configured to rigidly hold the at least one reservoir and the deformable tray. Additionally or optionally, the sleeve is frictionally engaged with the cartridge, and the cartridge is slidable relative to the sleeve upon receipt of a force sufficient to exceed the frictional engagement. In still other aspects of the present invention, the drug delivery device further includes a removable guard which blocks sliding movement of the cartridge relative to the sleeve. Additionally or optionally, the deformable tray forms an airtight seal at an end of the sleeve.

In certain aspects of the present invention, the at least one cavity comprises a plurality of cavities and the at least one reservoir comprises a plurality of reservoirs. Additionally or optionally, the plurality of cavities are arranged annularly in the tray.

In certain aspects of the present invention, the deformable tray is formed at least in part from an elastomeric material. Additionally or optionally, the lower surface of the tray is configured to partly adhere to skin of a subject of the drug delivery device. Still further, in some aspects of the present invention, the drug delivery device further includes a seal covering the lower surface of the tray.

In accordance with another aspect of the present invention, a method of administering a drug to a subject with a drug delivery device is provided. The device has a post structure, a deformable tray defining at least one microneedle cavity, and at least one cylindrical reservoir axially aligned with the at least one microneedle cavity. The methods includes steps of positioning the drug delivery device relative to the subject such that a lower surface of the deformable tray contacts skin of the subject; and moving the post structure relative to the deformable tray such that at least one post of the post structure moves toward an upper surface of the tray, through the at least one cylindrical reservoir, and into at least one microneedle cavity so as to exert a force on contents of the at least one reservoir and to exert a force on a microneedle in the at least one microneedle cavity sufficient to drive the contents and the microneedle through the lower surface of the tray.

In certain aspects of the present invention, the step of positioning the drug delivery device relative to the subject further includes at least partly adhering the lower surface of the deformable tray to the skin of the subject.

In some aspects of the present invention, the method of administering the drug to the subject with the drug delivery device includes before the positioning step, removing a seal covering the lower surface of the tray.

In still other aspects of the present invention, the post structure is frictionally engaged relative to the deformable tray, and the moving step includes applying a force sufficient to exceed the frictional engagement to the post structure. Additionally or optionally, the method of administering the drug to the subject with the drug delivery device includes before the moving step, removing a guard that blocks movement of the post structure relative to the deformable tray.

In accordance with yet another aspect of the present invention, a method of filling a drug delivery device is provided. The device has a cartridge and a deformable tray defining at least one microneedle cavity. The method includes steps of loading a liquid comprising the drug into at least one cylindrical reservoir positioned within the cartridge; and allowing the liquid to flow to the at least one microneedle cavity such that the drug is absorbed by at least one microneedle in the at least one microneedle cavity.

In some aspects of the present invention, the loading step includes loading the liquid having a volume of less than three times the volume of the at least one microneedle cavity.

In certain aspects of the present invention, the loading step includes inserting the liquid into an opening in an upper end of the cartridge.

In still other aspects of the present invention, the loading step includes immersing the cartridge in the liquid.

In some aspects of the present invention, the allowing step includes promoting absorption of the drug by the at least one microneedle using at least one post in the cartridge positioned above and spaced from the at least one cavity.

In certain aspects of the present invention, the method of filling the drug delivery device includes freeze drying the drug delivery device.

In still other aspects of the present invention, the method of filling the drug delivery device includes forming the at least one microneedle in the at least one microneedle cavity.

Aspects of the invention include a drug delivery device. The drug delivery device includes a post structure having a base and at least one post protruding from the base. The drug delivery device also includes a deformable tray movably coupled to the post structure. The deformable tray defines at least one cavity extending from an upper surface of the tray toward a lower surface of the tray. The at least one cavity tapers to a point at or near the lower surface of the tray. The drug delivery device also includes at least one microneedle positioned within the at least one cavity. The drug delivery device further includes at least one reservoir positioned directly above and axially aligned with the at least one cavity. The reservoir is configured to contain a therapeutic agent selected from lyophilized materials, spray-dried powders, spheronized particles, extruded implants, crystalline materials, and solid or semi-solid formulations. The drug delivery device also includes an adhesive surface positioned on the lower side of the deformable tray. The adhesive surface is configured to adhere to the skin and optionally remain on the skin when the microneedle device is withdrawn.

In some aspects of the invention, a method of administering a drug to a subject is provided. The method includes steps of: positioning a drug delivery device, such that a lower surface of a deformable tray contacts the subject's skin; and actuating a post structure to move at least one post toward an upper surface of the tray, through at least one reservoir, and into at least one microneedle cavity so as to exert a force on the contents of the at least one reservoir and drive the contents and the microneedle through the lower surface of the tray and into the subject's skin.

In still other aspects of the invention, a method of filling a drug delivery device is provided. The drug delivery device has a post structure, a deformable tray defining at least one microneedle cavity, and at least one reservoir axially aligned with the at least one microneedle cavity. The method includes steps of: immersing the reservoir structure in a liquid drug formulation or contacting the reservoir structure with a larger volume of drug formulation; and allowing the liquid to fill the at least one reservoir to a predetermined volume, defined by the fixed volume of the reservoir structure.

### BRIEF DESCRIPTION OF THE FIGURES

FIG. 1 shows an exploded view of an exemplary embodiment of the drug delivery device.

FIG. 2 shows a sidelong cutaway view of the exploded view of FIG. 1.

FIG. 3 shows a sidelong cutaway view of an assembled drug delivery device in accordance with an exemplary embodiment of invention.

FIG. 4 depicts an exemplary method of administering a drug to a subject with the drug delivery device of FIG. 1.

FIG. 5 depicts an exemplary method of filling the drug delivery device of FIG. 1.

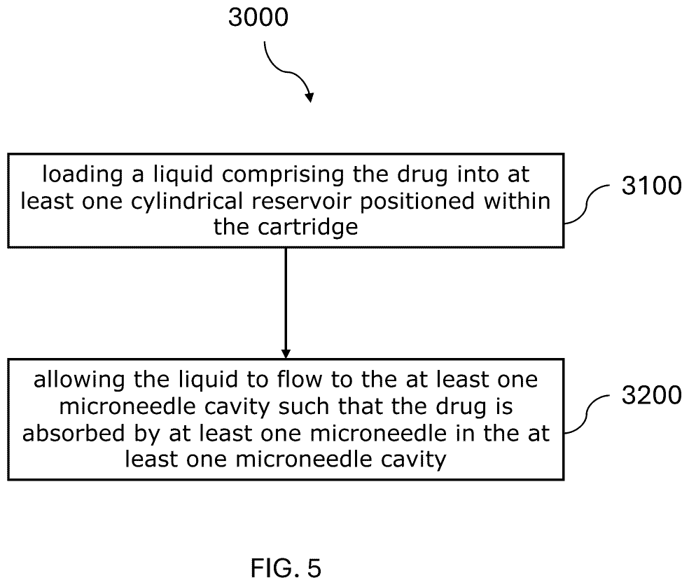

FIG. 6 depicts an exploded view of an exemplary embodiment of the drug delivery device.

FIG. 7 depicts an exemplary method of administering a drug to a subject.

FIG. 8 depicts an exemplary method of filling a drug delivery device.

### DETAILED DESCRIPTION OF THE INVENTION

The present invention involves solid, dissolvable or resorbable microneedle technology, in which the intended purpose is to deliver a dose of an active ingredient through the surface of a tissue target, depositing it therein for subsequent absorption and activity. It should be noted that a needle structure of the microneedle technology may not “dissolve” except due to gradual enzymatic erosion or self-degradation, for example. In this way, the needle structure(s) remain in the subject's skin and is/are not removed after delivery of the dose to the target tissue.

Skin is a resilient, flexible, viscoelastic tissue. When a pointed object is pressed against skin, it deforms, stretching and compressing to accommodate the pressure until the point begins to cut and penetrate the skin. The viscoelasticity of skin may be exploited in that the velocity of the needle structure can effectively reduce the force required for penetration, thus many MAPs are marketed with an associated spring-loaded applicator to accomplish this. While the degree to which the skin deforms is dependent upon the sharpness of the object and the velocity with which it is applied, conventional MAPs do not penetrate to their full needle length, substantially reducing the utilization of material and thus their delivery capacity.

A principal obstacle to increasing the penetration of microneedles in MAPs is the stretching and compressive deformation of skin. It is readily observable by pressing a finger against the skin of a human arm, that the skin indents, and further stretches laterally toward the contact point from areas adjacent to that point. In the case of a hypodermic injection, this effect increases the amount of force and distance the needle has to travel in order to penetrate the skin. For this reason, trained practitioners commonly stretch and stabilize the skin with one hand before performing the injection with the second hand, reducing the required force and the associated discomfort to the patient.

Similarly, skin under the points of microneedles indents and stretches in response to pressure, effectively distributing the applied force across a larger area. However, in conventional MAPs, the microneedles are attached to a fixed, approximately planar backing, which effectively stops any deeper penetration upon the skin contacting that backing. While deformation can be limited to some degree by use of a high-velocity applicator, such devices increase complexity and the inherent abrupt sound and skin impact can be startling or uncomfortable for patients. Unfortunately, the pointed, roughly pyramidal or conical structure of most microneedles therefore dictates that the amount delivered may therefore correspond to as little as 25% of the total volume of the microneedle. For this reason, drug loading in conventional MAPS generally is localized to the tip portion of the needle structures, both restricting overall material utilization and requiring highly specialized methods and equipment to place a drug payload precisely onto these microscopic features.

The area of a MAP is effectively limited by the non-planarity of most body surfaces. In practice, it is challenging to apply MAPs with sufficient force to penetrate the skin if they are larger than a few centimeters across. When MAPs are used for vaccination or contraception purposes, especially in Low- and Middle-Income Countries (LMIC), cultural norms may further limit what areas of skin may be appropriately accessed. Maximizing the dose delivered in a small area is thus a desirable characteristic of microneedle delivery systems.

Therefore, it will be apparent to one of ordinary skill in the art of drug delivery, even with little familiarity of microneedle-mediated delivery, that existing MAP technology is limited by payload delivery capability, and that a need exists for improved payload delivery and that such an improvement would be of benefit and value.

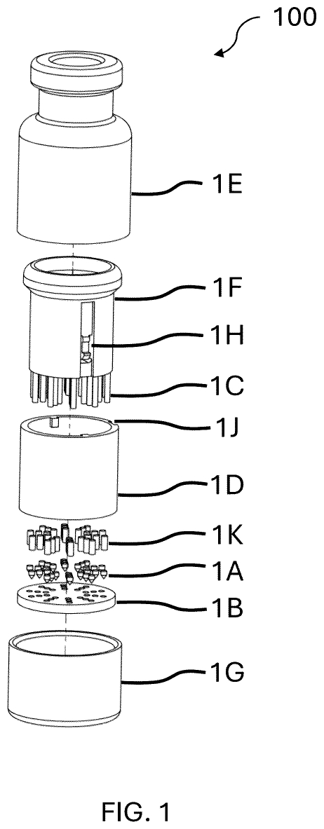

Referring generally to the figures, FIG. 1 is an exploded view of the exemplary components of a drug delivery device 100 in accordance with an exemplary embodiment of the invention. Exemplary components include freestanding microneedle structures 1A; a deformable base 1B; post structures 1C; a reservoir structure assembly 1D; an enclosure 1E; an integrated support 1F; a removable guard ring 1G; minimum force trigger 1H; a plurality of alignment features 1J; and payloads 1K contained by the reservoirs 1D. Enclosure 1E includes an upper exterior portion simulating a vial and providing a means to grip the device 100 in use, as well as a sterile barrier following installation of a crimped-seal cap and stopper. Integrated support 1F provides support for the post structures 1C providing alignment features 1J and a central channel through which a vaccine dose can pass through to the reservoir channels. Removable guard ring 1G is configured to prevent accidental deployment and provides a sterile barrier at the bottom of the device 100. Minimum force trigger 1H is configured to ensure sufficient force is applied along the device axis before the device 100 is actuated to deliver the dose. Alignment features 1J are configured to ensure that posts 1C, reservoirs 1D, microneedles 1A and the functional assembly are retained in correct alignment.

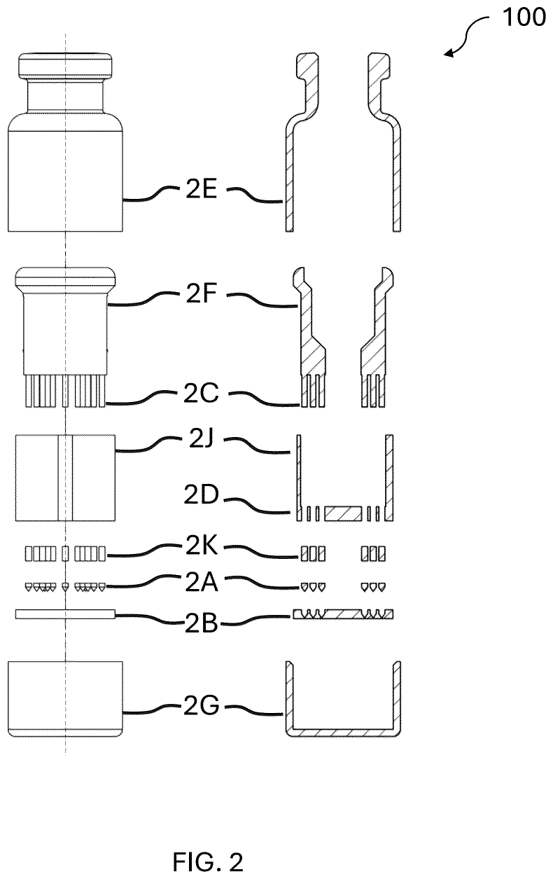

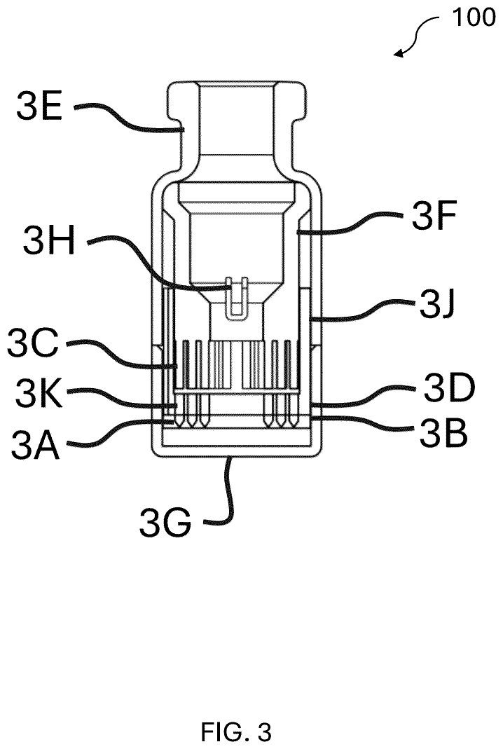

FIG. 2 shows a sidelong cutaway view of the exploded components of the device 100 illustrated in FIG. 1, such that the individual labeled parts of FIG. 2 are matched to those of FIG. 1, e.g., that microneedles 2A in FIG. 2 correspond to microneedles 1A in FIG. 1. Likewise, FIG. 3 shows a sidelong cutaway view of an assembled version of the device 100, such that the components identified in FIG. 3 corresponds to the components in FIGS. 1 and 2.

### Vaccines

Aspects of the present invention are directed to intracutaneous vaccine administration by microneedles 1A, 2A, 3A of drug delivery device 100 versus the traditional intramuscular injection route. The benefits and advantages associated with such vaccine administration are myriad, and include: dose-sparing/utilization efficiency; increased thermal stability; improved patient acceptability; weight and logistics reduction; avoidance of reconstitution errors and needle-stick injuries, and reduced provider training requirements, to name a few. Microneedle-mediated vaccination could be of great benefit in global health. It should be noted, however, that cost is an important factor in mass-vaccination efforts, and the cost of materials, manufacture, transportation, loss to instability, and administration are all limiting to the success of such programs. Therefore a microneedle delivery technology that may be produced and administered at a reduced cost would be of particular benefit in LMIC vaccine efforts.

### Microneedle Loading

A principal limitation of conventional microneedle-mediated delivery is that the total volume of material delivered is limited to the very small dimensions of the microneedle tips. Loading microneedles with liquid serum can involve highly specialized equipment and processes that are not compatible with, nor easily transitioned into, existing vaccine industrial production for LMIC. Development of specialized loading methods adds cost and delays implementation of MAPs in global health efforts, despite other advantages noted for MAP vaccine administration.

Existing LMIC vaccines are mass-produced on high-speed aseptic filling lines, using standardized equipment, such as, the FLC 3000 filler (Syntegon Technology GmbH, Germany). These systems are capable of dispensing liquid vaccines into hundreds of vials a minute, and typically vials are fed directly into lyophilization units and then capped, all in the context of a highly-automated aseptic manufacturing process. The minimum dispense quantity of the FLC 3000 for example is 200 microliters, while MAP needle structures dimensionally constrain payload capacity to many orders of magnitude lower volumes. A 200 microliter volume of vaccine serum as currently manufactured would entirely immerse most demonstrated microneedle products, and would be impossible to constrain to the penetrating portions of the structures. Thus, existing microneedle patches are loaded with custom equipment, precisely dispensing much smaller volumes of vaccine serum at a much higher concentration, in a manner that is not realistically achievable using existing commercial vaccine manufacturing/filling equipment designed for glass vials, the overwhelmingly predominant form of vaccine distribution.

Therefore, while microneedle delivery systems may pose real benefits for LMIC vaccines, they are very poorly suited for integration into current vaccine manufacturing processes, for several reasons. Accordingly, it will be apparent to one of ordinary skill in the art of drug delivery that adoption of existing MAP technology is limited by incompatibility with existing, industry-standard automated filling processes and equipment, and that a need exists for improving conventional MAPs to make them compatible for use with such processes and equipment, and that such an improvement would be of benefit and value.

### Freestanding Microneedle Structures

With reference to the drawings, FIGS. 1 and 2 depict exploded views of an example drug delivery device 100 according to one aspect of the present invention. The drug delivery device 100 makes use of pre-formed microneedle structures 1A, 2A, 3A positioned in a deformable or elastomeric base layer 1B, 2B, 3B, with cavities in the base 1B matching the microneedles 1A, 2A, 3A. This cavity in the base 1B, 2B, 3B may be used to form molded microneedles 1A, 2A, 3A by solidifying a liquid material in the cavities. In some embodiments the needle structures 1A, 2A, 3A may be formed in this manner. In other embodiments they may be pre-formed and separately introduced to the cavities. The cavities can all be similarly oriented such that the base 1B, 2B, 3B can be placed atop a surface in a manner that positions all of the microneedle 1A, 2A, 3A points toward that surface. In one embodiment the elastomeric base layer 1B, 2B, 3B is typically only slightly thicker than the height of the needle 1A, 2A, 3A (the distance between the pointed tip and the base). In another embodiment the elastomeric layer 1B, 2B, 3B forms a continuous sterile barrier across the sharp tips of the microneedles 1A, 2A, 3A. In a preferred embodiment, the base layer 1B, 2B, 3B comprises an array of microneedles 1A, 2A, 3A approximately 1 mm in height, spaced in a regular pattern wherein the microneedles 1A, 2A, 3A are spaced on centers, each pitched at approximately 1.5 mm distant from adjacent needles 1A, 2A, 3A. In a further preferred embodiment, the needle structures 1A, 2A, 3A are composed of materials with sufficient rigidity relative to the deformable base 1B, 2B, 3B that when the needles 1A, 2A, 3A are pressed into the base, the sharp tips cut through the deformable base 1B, 2B, 3B, and can be driven completely through the base 1B, 2B, 3B without substantially damaging the needles 1A, 2A, 3A. While any bioresorbable material of sufficient rigidity may be conceivably used to form the microneedle structures 1A, 2A, 3A, exemplary materials include hydrocolloids, polylactic or polylactic-co-glycolic acids or similar polyhydroxyalkanoates, polyvinyl pyrrolidone, sugars, or any of the many other suitable materials of construction in the field of bioresorbable microneedle delivery systems. Without being bound to any particular composition or method of manufacture, materials may generally be thermoformed, molded from solutions, produced as fibers, polymerized in situ, or solidified by any of the processes already established to create molded microneedles 1A, 2A, 3A, or separately formed and afterwards combined with the base layer 1B, 2B, 3B, or the base layer 1B, 2B, 3B may be molded over a set of pre-formed microneedles 1A, 2A, 3A.

In some embodiments, the needle 1A, 2A, 3A components may further comprise a drug, biologic, or other material included to provide a benefit. In other embodiments, a drug or other active ingredient may be wetted, coated, or otherwise co-delivered with the needle structures 1A, 2A, 3A.

### Skin Engagement

The freestanding microneedle component 1A, 2A, 3A according to aspects of the present invention can provide a surprising and unexpected benefit in the form of enhanced skin penetration when the needles 1A, 2A, 3A are pressed through the deformable base layer 1B, 2B, 3B. This benefit may arise from engagement of the skin by the inherent traction afforded by the contact between the skin and the material of the deformable base layer 1B, 2B, 3B that limits the degree to which the skin could accommodate in response to the force applied at the needle tip of microneedles 1A, 2A, 3A. In this respect, the frictional interaction between the skin and the base 1B, 2B, 3B may be configured to interfere with the lateral stretching described above. Further, as needles 1A, 2A, 3A press through the base 1B, 2B, 3B, the base 1B, 2B, 3B must itself stretch laterally, potentially engaging the local underlying skin in a manner similar to the manner in which health care providers stretch skin prior to performing an injection. Due at least in part to the above features, a dramatic and surprising enhancement of needle penetration was noted with full length needles delivered millimeters below the skin surface with unexpectedly low force, e.g. to a length of 60%, 70%, 80%, 90%, 100%, or over 100% of the microneedle 1A, 2A, 3A, beyond what would typically be achievable in the context of a conventional MAP device.

In some embodiments, the deformable base material 1B, 2B, 3B and surface is preselected to have characteristics matched to maximally engage skin, with a sticky or tacky texture, a surface pattern, or a coating of an additional adhesive material, for the purpose of enhancing skin penetration and reducing the required force to achieve this.

### Post Structures

With reference to the example drug delivery device 100 depicted in FIGS. 1 and 2, drug delivery devices 100 according to aspects of the present invention can make use of post structures 1C, 2C, 3C to drive the pre-formed microneedles 1A, 2A, 3A through the elastomeric base 1B, 2B, 3B, and into the underlying surface, for example skin. In this embodiment, the posts 1C, 2C, 3C are positioned vertically above the microneedle structures 1A, 2A, 3A such that they are configured to pass readily through the upper opening of the base layer 1B, 2B, 3B. In one embodiment the post structures 1C, 2C, 3C are approximately of the same cross-sectional dimension as the microneedle 1A, 2A, 3A bases. In other embodiments, the cross-section of the posts 1C, 2C, 3C may be larger or smaller than the microneedle bases 1B, 2B, 3B, or may taper. In a preferred embodiment, the post structures 1C, 2C, 3C are taller than the microneedle structures 1A, 2A, 3A and the deformable base 1B, 2B, 3B thickness such that the posts 1C, 2C, 3C can press the microneedles 1A, 2A, 3A fully through the base layer 1B, 2B, 3B, and drive them a predetermined distance (e.g. at least 2 millimeters) past the bottom face of the base layer 1B, 2B, 3B, embedding them below the upper surface of the underlying skin. In another embodiment, the posts 1C, 2C, 3C may be integrally formed with or mounted onto a monolithic backing layer permitting them to be pressed together as a unit through the microneedle base layer 1B, 2B, 3B.

### Reservoir Channel Structures

With reference to the example drug delivery device 100 depicted in FIGS. 1 and 2, drug delivery devices 100 according to aspects of the present invention can include an additional layer or plate containing reservoir channels or voids may be included between the posts 1C, 2C, 3C and the microneedle base 1B, 2B, 3B. The channels of the reservoir structure assembly 1D, 2D, 3D may be loaded with a drug, vaccine, or other material, which may be referred to herein as the Payload 1K, 2K, 3K. These channels may be oriented in alignment with the posts 1C, 2C, 3C, such that the posts 1C, 2C, 3C can be driven through the channels to compress and/or eject their contents. In one embodiment, the channels are situated, each below a post structure 1C, 2C, 3C and also above a needle structure 1A, 2A, 3A, such that when the posts 1C, 2C, 3C are pressed, the contents of the reservoir 1D, 2D, 3D are compressed onto the bases 1B, 2B, 3B of the underlying microneedle structures 1A, 2A, 3A, and when the posts 1C, 2C, 3C are pressed further, the microneedles 1A, 2A, 3A are driven through the base layer 1B, 2B, 3B with a matching mass or pellet of compressed Payload 1K, 2K, 3K material, such that the microneedle 1A, 2A, 3A and Payload 1K, 2K, 3K are simultaneously driven into the underlying skin. In some embodiments the Payload 1K, 2K, 3K is a compressed and/or lyophilized vaccine or other therapeutic agent. In another embodiment the reservoir channel plate contains reservoirs 1D, 2D, 3D with a total volume sufficient to accept a 200 microliter vaccine serum dispensed by an automated filling machine as described above. In other embodiments the reservoirs 1D, 2D, 3D are of dimensions optimized to self-fill by capillary action and the plate may include features that aid in distributing a dispensed volume to the full set of reservoirs 1D, 2D, 3D. In a preferred embodiment, the reservoir plate is separately loaded with vaccine serum and lyophilized such that the channels are pre-loaded with lyophilized Payload material. In some embodiments of the present invention, the reservoir channels are a necessary component, contributing the capacity to load the assembly with larger volumes of a drug product composition. In other embodiments, the channels are an optional feature, and can be entirely omitted without negative effect upon the capability to deliver the microneedle tip structures 1A, 2A, 3A into skin by the action of the post assembly 1C, 2C, 3C.

In some embodiments of the present invention, the reservoir channels are a necessary component, contributing the capacity to load the assembly with larger volumes of a drug product composition, especially relevant to vaccine production processes. In other embodiments, the channels are an optional feature, and can be entirely omitted without negative effect upon the capability to deliver the microneedle tip structures into skin by the action of the post assembly. In other embodiments, multiple reservoir plates may be stacked to co-administer multiple materials, or the reservoir channels may be co-loaded loaded with multiple discrete payloads. In other embodiments the payloads may be liquid at body temperature, but maintained in a solid state until injected by controlling the device temperature. In other embodiments, the payloads may comprise bioerodible implants or compositions for sustained release. In one embodiment, payloads 1K, 2K, 3K may be selected to co-administer a primary vaccine dose and a booster dose as a secondary payload comprising a delayed release composition.

### Actuation and Device Characteristics

As shown in FIGS. 1, 2, and 3, the posts 1C, 2C, 3C, reservoirs 1D, 2D, 3D, and microneedle tips 1A, 2A, 3A may further be assembled into an integrated device, maintaining them in fixed orientation and alignment suitable for use as a drug delivery device 100. As shown in FIG. 1, some embodiments of the device 100 may contain additional features to maintain fixed orientation during deployment, as well as trigger mechanisms 1H, 3H that allow the device 100 to be deployed only when sufficient force is applied, to ensure successful penetration of the needles and the Payload 1K, 2K, 3K into the skin. Other features in preferred embodiments may further include visual, tactile, and auditory feedback, safety features to prevent unintentional deployment, and sterile barriers to prevent contamination or degradation of the payload 1K, 2K, 3K or components during transport or storage prior to use.

In one preferred embodiment the device 100 can be transported and stored as a sterile assembly in the absence of the reservoir structures, but can be fitted with pre-filled reservoirs at point-of-use, such that the Payload may be configured specific to the momentary need. Similarly, stockpiles of pre-loaded reservoir plates may be manufactured and then distributed along with stockpiled assemblies as part of a rapidly configurable response to emerging public health needs.

### Loading of Pre-Assembled Devices

In some embodiments, the device 100 can be assembled with empty reservoir channels and then filled and lyophilized using existing automated vaccine processing infrastructure. Lyophilization, or freeze-drying, of vaccines and similar Payload materials is reliant upon effectively reducing the temperature of an aqueous liquid dose to below freezing point, and then precisely controlling the temperature as the water is sublimed from the frozen material. In some embodiments, the device 100 is constructed of materials with specific pre-selected thermal conductivity. In some embodiments, the device 100 is constructed of injection molded resins doped with thermally conductive materials to achieve specific performance in lyophilization processes.

In one preferred embodiment, the posts 1C, 2C, 3C, microneedles 1A, 2A, 3A, and reservoirs 1D, 2D, 3D are configured such that dose of liquid vaccine or other benefit agent can be dispensed into the assembly through a channel, such that the liquid is selectively directed into the reservoir channels. In some embodiments the liquid may be directed to the reservoir channels by the influence of gravity, selective wetting of surfaces, surface tension, capillary action or other means, or by a combination of design and material characteristics designed to accomplish this

In one preferred embodiment, the device 100 is pre-assembled in a form that mimics existing vaccine vials, for the purposes of processing in existing filling lines. In this form, the external dimensions of the device 100 match those of a vial, for example a standard 2R vial, for example as specified in ISO 8362-1:2018. The materials of composition and internal structure may further be preselected to ensure that the center of mass of the device 100 is compatible with the dynamics of the vial handling process in such filling lines. Further, the exterior of the device 100 may be constructed to accommodate the stoppering and crimped cap sealing process of existing lines. Such adaptations of the present invention may be adopted to maximize compatibility with existing vaccine infrastructure, reducing barriers to rapid and widespread implementation. In another preferred embodiment, the upper exterior portion of the device 100, as best shown in FIG. 1E, may be readily gripped to apply appropriate pressure to actuate the mechanism, thus driving the microneedles and associated payload 1K, 2K, 3K into the underlying skin.

Compatibility with Commercial Vaccine Vial-Filling Processes

In order to avoid the challenges of complex tip-loading approaches, or the need for highly non-standard concentrated vaccine sera or processing sera directly into microneedle structures, some embodiments may include assembling the freestanding microneedles 1A, 2A, 3A described above into a vial-like structure that could be processed on an existing commercial vaccine automated filling line. In such embodiments, the rod structures positioned over the needle structures 1A, 2A, 3A in the elastomeric mold can be provided in an integrated assembly of dimensions that would fit into a standard “2R” vial (per International Organization for Standardization (ISO) 8362-4:2011) footprint, such that a vial-shaped product could be handled in existing automated fill-lines and loaded with minimal modification to existing industrial equipment, formulations, and processes. The 2R vial footprint limits the area of the elastomeric needle mold structures of the present invention to an area corresponding to the 2R vial base, a circle of 16 mm diameter. This area limitation in turn limits the number of needle structures 1A, 2A, 3A that can be formed in this area. For example, an efficient hexagonal pattern of needle structures of 1.0 mm diameter at the base, spaced with gaps between needles of approximately 0.5 mm, permits a maximum of about 61 such needles distributed across the base of a 2R vial footprint. The distribution, spacing, and volume of microneedles 1A, 2A, 3A may be selected based on production fill requirements and dosing requirements for the particular vaccine.

In a configuration mimicking the interior of the 2R vial, the post structures 1C, 2C, 3C that provide compression and actuation, pushing the needles 1A, 2A, 3A and associated vaccine solids into the underlying skin can be maintained in fixed positions relative to each other and to their respective reservoirs 1D, 2D, 3D and needles 1A, 2A, 3A. The fixed spatial relation between the individual posts 1C, 2C, 3C is accomplished by joining the posts 1C, 2C, 3C at the ends distal from the needles 1A, 2A, 3A, mounted or co-molded in a rigid plate or similar monolithic structure. Thus fixed, the post structures 1C, 2C, 3C can be simultaneously deployed in the same trajectory down through the reservoir channels 1D, 2D, 3D and the elastomeric layer, depositing the needles 1A, 2A, 3A and the vaccine payloads 1K, 2K, 3K into the skin. The fixed spatial relation between the post assembly 1C, 2C, 3C, the reservoir channels 1D, 2D, 3D, and the elastomeric base layer 1B, 2B, 3B can be accomplished by seating these three components into a rigid sheath, which can be made of any material suitable for vaccine contact, but in one embodiment is made of a standard syringe material such as described in ISO 11040-6:2012, for example polypropylene or a cyclo-olefin polymer. This rigid sheath 1F, 2F, 3F is formed with an alignment feature, for example, at least one raised internal ridge or guide structure running along its long axis, keyed to match at least one corresponding slot feature in the post assembly, the reservoir channel plate, and the elastomeric base plate. The posts 1C, 2C, 3C, reservoir plate, and elastomeric base 1B, 2B, 3B are assembled so that their slots are fitted into the internal guide ridge of the sheath, and thus can be maintained in strict alignment with each other. The reservoir plate and the elastomeric base 1B, 2B, 3B are affixed or tightly fitted to the sheath structure, with the elastomeric base 1B, 2B, 3B further molded to form an airtight seal at one end of the sheath and the reservoir plate seated directly against the base. The assembled Actuator includes the post assembly 1C, 2C, 3C, the sheath 1F, 2F, 3F, the reservoirs 1D, 2D, 3D, and the elastomeric mold containing the needle tip structures. Reference to the Actuator below generally will assume that the removable guard component 1G, 2G, 3G is in place, and it would only be removed to administer the dose.

### Heat Transfer During Lyophilization

Industrial lyophilization chambers used in vaccine manufacture are commonly constructed such that stainless steel shelves contain cooling elements or recirculated coolant channels, and filled vials are placed in direct contact with the cooled shelves. Effective lyophilization requires that the vial contents are cooled to freezing temperature such that water is sublimed from the solid material rather than evaporating from a liquid surface, creating a porous solid that may inhibit protein aggregation and is amenable to rapid dissolution when reconstituted. To be effectively lyophilized using existing industry processes, the materials of the present invention may be selected such that heat transfer occurs on a similar timescale as for existing glass vial formats. Glass has a thermal conductivity value of about 1.2 W/m-K, whereas silicones and thermoplastics commonly possess a much lower thermal conductivity value of around 0.2 W/m-K, although specific materials may vary. Therefore, substitution of the actuator components for the glass bottom of a standard 2R vial would potentially lower the heat transfer rate and negatively impact the lyophilization process or substantially extend process time needed for this manufacturing operation. Fortunately, high thermal conductivity elastomers and thermoplastics are available, which commonly make use of filler materials such as aluminum oxide and boron nitride, which impart much higher thermal conductivity to the bulk materials, matching or exceeding that of glass. In some embodiments, such high thermal conductivity polymer systems can be used, especially for the base layer and the reservoir layer of the actuator, where heat transfer to the underlying cooled shelf is most relevant to the lyophilization process.

### Use of Standard Glass Cartridges for Pre-Filled Syringes

In accordance with one aspect of the present invention, the drug delivery device 100 is made compatible with the automated fill, lyophilization, and crimp-sealing processes of large scale vaccine manufacture. To this end, the post driven deposition device can be integrated into a standard glass cartridge that is essentially a 2R glass vial without a bottom (e.g., provided by Schott Pharma AG & Co KGaA, Germany). Such cartridges can be used as components of pre-filled syringe devices, and the open end can be fitted with a plunger rod and stopper to create a syringe configuration. These cartridges may be filled and processed in a manner very similar to vial-filling automated fill lines. In accordance with aspects of the present invention, the space normally used for the syringe plunger is instead fitted with an actuator.

The actuator may be situated within the glass cartridge such that the post component is in contact with the shoulder of the glass cartridge. The remaining parts of the actuator can be designed to move as one piece, relative to the post component, during vaccine administration.

In one exemplary operation, during filling and subsequent processing, the device 100 is constrained to a specific, non-actuated position by the presence of a guard ring 1G, 2G, 3G, affixed to the exterior of the device 100, that prevents accidental deployment of the device 100 until the guard ring 1G, 2G, 3G is removed. Further the contact between the actuator and the cartridge interior may be maintained by a ring of material that acts in a manner similar to the stopper in a standard prefilled syringe, maintaining a sterile barrier to the interior of the cartridge, this can be an elastomeric O-ring or co-molded seal. In some embodiments this ring 1G, 2G, 3G might be replaced with a viscous fluid such as a silicone lubricant or petroleum jelly, so long as this is capable of maintaining an air-tight seal. In some embodiments, the seal ring 1G, 2G, 3G may serve a secondary purpose of providing an initial resistive force or trigger, or an additional ridge or structure may be included for that purpose. The outer surface of the elastomeric mold at the bottom of the actuator may also be fitted with a secondary barrier of, for example, aluminized mylar, similarly preventing ingress of contamination to the sterile interior space. Such secondary barriers would be removed along with the guard before use.

In some embodiments, the post component 1C, 2C, 3C may include a central void or funnel feature, such that during filling, the vaccine serum delivered through the mouth of the cartridge may travel unobstructed to the reservoir channels. Once in contact with the upper surface of the reservoir channel plate, the serum may be spontaneously drawn into the channels by a combination of gravity, capillary action, wetting of the channel interior, surface tension, and the venting of displaced air through for example fine groove or channel structures between the reservoir plate and the elastomer base.

### Suitability for Mass-Production

The above description of the present invention addresses a variety of issues involved in bridging the technical constraints that make present-day automated aseptic vaccine fill/finish manufacturing lines incompatible with the loading requirements of the current generation of microneedle delivery devices. By providing a technically feasible interface that addresses form-factor, dispense volume, heat transfer during lyophilization, and general compatibility with aseptic fill processes and equipment, and simultaneously addressing volume limitations, delivery constraints, and skin penetration dynamics, the inventor hopes to contribute to the increased availability of microneedle-mediated vaccines for global health, and especially LMIC vaccine availability.

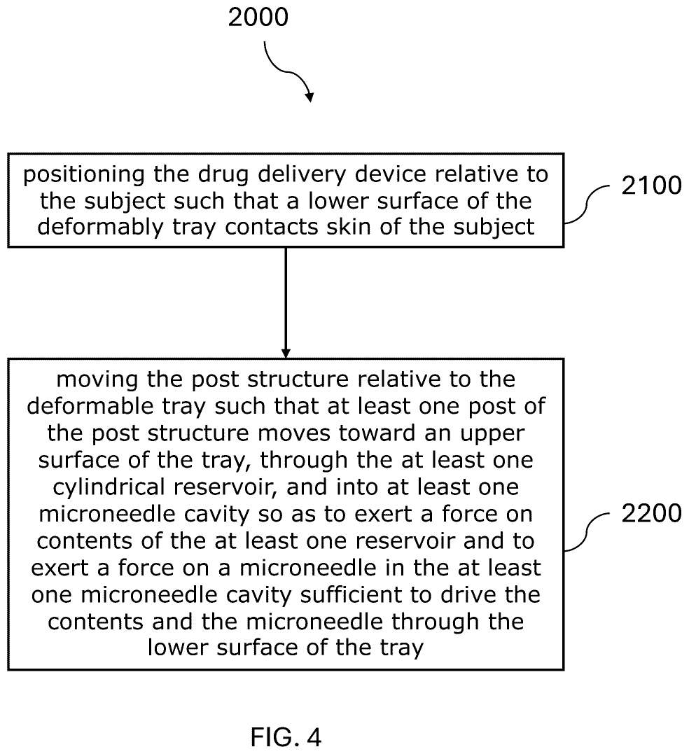

Turning now to FIG. 4, in an exemplary embodiment, a method of administering a drug to a subject with a drug delivery device 100, such as method 2000, is provided. The device 100 has a post structure 1C, 2C, 3C, a deformable tray 1B, 2B, 3B defining at least one microneedle cavity, and at least one cylindrical reservoir 1D, 2D, 3D axially aligned with the at least one microneedle cavity. The methods includes: step 2100 of positioning the drug delivery device 100 relative to the subject such that a lower surface of the deformable tray 1B, 2B, 3B contacts skin of the subject and step 2200 of moving the post structure 1C, 2C, 3C relative to the deformable tray 1B, 2B, 3B such that at least one post 1C, 2C, 3C of the post structure moves toward an upper surface of the tray 1B, 2B, 3B, through the at least one cylindrical reservoir 1D, 2D, 3D, and into at least one microneedle cavity so as to exert a force on contents of the at least one reservoir 1D, 2D, 3D and to exert a force on a microneedle 1A, 2A, 3A in the at least one microneedle cavity sufficient to drive the contents and the microneedle 1A, 2A, 3A through the lower surface of the tray 1B, 2B, 3B.

In an exemplary embodiment, step 2100 further includes at least partly adhering the lower surface of the deformable tray 1B, 2B, 3B to the skin of the subject. In a non-limiting example, method 2000 includes step 2300 of removing a seal covering the lower surface of the tray 1B, 2B, 3B, before performing step 2100. In still another non-limiting example, the post structure 1C, 2C, 3C is frictionally engaged relative to the deformable tray 1B, 2B, 3B, and step 2200 includes applying a force sufficient to exceed the frictional engagement to the post structure 1C, 2C, 3C. Additionally or optionally, method 2000 includes, before performing step 2200, a step 2400 of removing a guard that blocks movement of the post structure 1C, 2C, 3C relative to the deformable tray 1B, 2B, 3B.

In another exemplary embodiment, a method of filling a drug delivery device 100, such as method 3000, is provided. The device 100 has a cartridge and a deformable tray 1B, 2B, 3B defining at least one microneedle cavity. Method 3000 includes a step 3100 loading a liquid comprising the drug into at least one cylindrical reservoir 1D, 2D, 3D positioned within the cartridge and step 3200 of allowing the liquid to flow to the at least one microneedle cavity such that the drug is absorbed by at least one microneedle 1A, 2A, 3A in the at least one microneedle cavity.

In a non-limiting example, step 3100 includes loading the liquid having a volume of less than three times the volume of the at least one microneedle cavity. In another non-limiting example, step 3100 includes inserting the liquid into an opening in an upper end of the cartridge. In still another non-limiting example, step 3100 includes immersing the cartridge in the liquid.

In a non-limiting example, step 3200 includes promoting absorption of the drug by the at least one microneedle 1A, 2A, 3A using at least one post 1C, 2C, 3C in the cartridge positioned above and spaced from the at least one cavity. In another non-limiting example, method 3000 includes freeze drying the drug delivery device. Additionally or optionally, method 3000 includes forming the at least one microneedle 1A, 2A, 3A in the at least one microneedle cavity.

In another embodiment, the present invention is directed to a microneedle-based delivery system that can (1) accommodate lyophilized and/or non-lyophilized materials, (2) provide controlled or sustained release of therapeutic agents, (3) integrate an adhesive layer capable of promoting skin hydration, forming a protective barrier, or acting as an independent transdermal delivery system, or a combination thereof. In an exemplary embodiment, the present invention is directed to a versatile microneedle system that integrates the above-listed features in a device suitable for mass-production using existing commercial aseptic filling lines. Still further, the present invention provides improved microneedle drug delivery devices and systems designed to enhance the versatility, payload capacity, and therapeutic applicability of microneedle-based drug delivery. The invention overcomes limitations in traditional microneedle array patches (MAPs) by enabling delivery of both lyophilized and non-lyophilized drug products, while also incorporating a unique adhesive tray that offers additional functionality.

Conventional microneedle-based drug delivery systems have emphasized the use of lyophilized formulations, which offer improved stability for temperature-sensitive biologics such as vaccines. However, a significant number of active pharmaceutical ingredients (APIs) are formulated as non-lyophilized products, such as spray-dried powders, spheronized particles, or extruded implants designed for sustained release. These materials offer distinct advantages in terms of stability, manufacturing efficiency, and controlled-release capabilities, yet existing microneedle systems have generally lacked the design flexibility to accommodate these formulations.

Conventional microneedle patches suffer from an inherent design limitation in that microneedle structures protrude from the surface of a backing layer, which imparts several constraints upon manufacture and use. When a sharp point is applied to skin, the skin responds via stretching deformation or “tenting” until sufficient force is generated to achieve cutting penetration. Microneedles must therefore be long enough to surpass the deformation limit of the skin and further insert the needle tips to the desired depth. The presence of a backing layer limits the degree to which a needle structure can be pressed into the skin, and needle length is limited by the aspect ratio of the material itself. While high velocity application can reduce the impact of these limitations in practice, non-uniform penetration by patch-based microneedles presents a substantial burden in design and implementation of these delivery platforms.

Furthermore, the process of microneedle penetration itself creates a transient disruption of the skin barrier, which presents an opportunity for enhanced topical or transdermal drug absorption. Conventional microneedle patches have generally not exploited this opportunity to co-administer therapeutic agents through the disrupted skin barrier, nor have they effectively combined microneedle-mediated delivery with complementary transdermal patch technologies.

Generally speaking, the present invention provides a drug delivery device 200 having freestanding microneedle structures 221 supported by a deformable, skin-contacting tray 220. The microneedles 221 are positioned points-downward within cavities of the tray 220 and are actuated by an array of posts 210a aligned above them. Deployment of the device 200 consists of moving the posts 210a downward against the back of the microneedle tips 221, driving them through the deformable tray 220 and into the underlying skin. Absent the backing layer of typical microneedle patches, the posts 210a are able to drive the needles 221 deeper into the skin. The deformable tray 220 may further be configured to adhesively engage the skin, thereby limiting the normal “tenting” deformation of skin during penetration and thereby increasing control and uniformity of the microneedle penetration.

In another aspect, the device 200 may be configured with reservoir channel plates 230, 240, which may be loaded with additional dose payload to be delivered. Upon deployment, the posts 210a compress the payload material 231, 241—stored in aligned reservoir chambers—and drive both the microneedles 221 and the payload 231, 241 into the skin with controlled force. In this way, the device 200 is capable of delivering both lyophilized and non-lyophilized compositions, including semi-solid formulations, extruded implants, or pre-formed tablets for immediate or sustained release.

In another aspect, the deformable tray 220 comprises an adhesive lower surface designed to engage the skin and, optionally, to remain in place after device 200 removal. This adhesive layer may provide one or more additional functions, such as maintaining localized hydration, forming a protective barrier, or delivering a secondary transdermal drug payload. By locally engaging and stabilizing the skin during needle insertion, the tray 220 reduces local skin deformation, providing more uniform and controlled insertion dynamics. These features enable combination therapies, prolonged delivery, and post-penetration care in a single application.

The invention further contemplates a modular approach to dosing, in which pre-filled reservoir components containing specific drug payloads may be inserted into the device at the time of use, enabling point-of-care selection of the therapeutic agent and dose responsive to clinical need. This configuration supports rapid customization and stockpiling of standardized components. In some embodiments, multiple reservoirs may be incorporated into a single device to enable co-administration of distinct therapeutic agents or temporally staggered doses within a single application.

In a further aspect, the device 200 is designed for compatibility with existing aseptic pharmaceutical filling infrastructure. In one embodiment, the device 200 conforms to ISO-standard vial formats, allowing integration with high-speed automated filling, lyophilization, and capping systems. In a non-limiting example, the device is dimensioned to fit the ISO-standard 2R vial footprint, thereby facilitating compatibility with high-speed industrial filling systems. The device 200 may be transported as a sterile, pre-assembled unit or loaded with payload material just prior to use. Alternative filling methods are also provided, including bulk filling by immersion or capillary action, simplifying manufacturing without compromising dose accuracy.

Collectively, the above-described features provide a platform for delivering a wide range of therapeutic agents, including vaccines, biologics, analgesics, hormones, antibiotics, and regenerative therapies. The invention offers particular advantages for global health applications by enabling efficient, scalable, and cost-effective production of microneedle-based drug products suitable for use in both advanced and resource-limited settings.

Thus, according to a specific aspect of the present invention, the microneedle drug delivery devices capable of accommodating a wide range of therapeutic agents, including non-lyophilized materials, implants, and sustained-release compositions. In a non-limiting example, the microneedle drug delivery device 200 may include a deformable tray 220 that adheres to the subject's skin, thereby forming a protective or hydrating barrier, or functioning as a secondary transdermal delivery system. Additional details of the drug delivery device 200 are discussed below, including with reference to potential configurations, materials, and manufacturing methods.

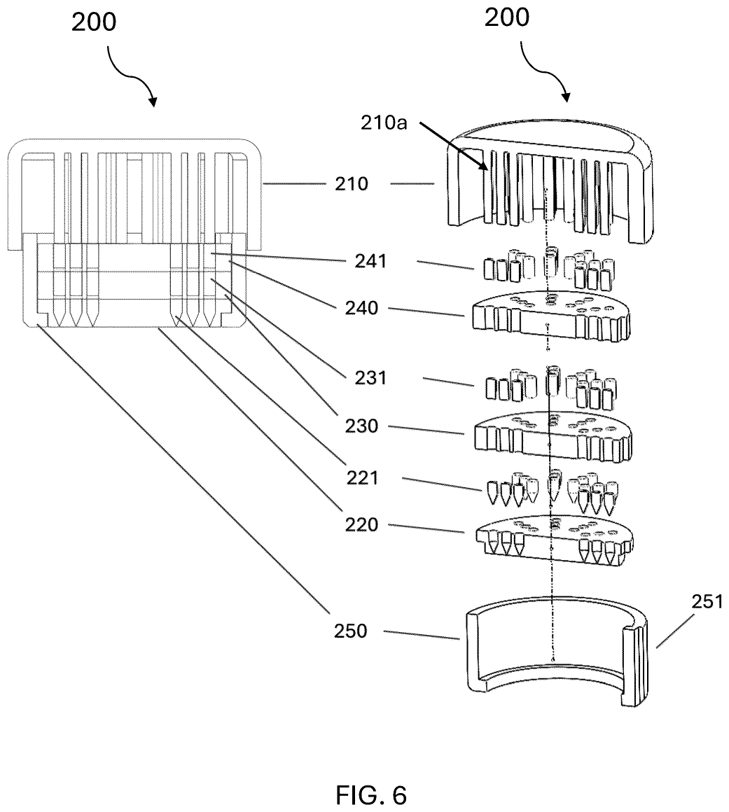

Referring now to FIG. 6, an exemplary embodiment of the device 200 in assembled form (left) and exploded view (right) is illustrated. The drug delivery device 200 includes at least one post or post assembly 210a. In a non-limiting example, the at least one post or post assembly 210a is formed integrally with a top housing cover 210. The posts and other components are positioned by alignment features 251 in a lower housing 250, such that they are directly above a matching set of free-standing microneedle tips 221 embedded in a deformable elastomeric tray 220. In this embodiment, two reservoir trays 230, 240 are shown, with their corresponding payloads 231, 241 situated within, all of which are in alignment with the posts 210a and microneedle tips 221. When the assembled device is positioned such that the elastomeric tray 220 is in contact with a skin surface, when downward pressure is exerted against the top housing cover 210, the posts 210a pass downward through the other components, compressing payloads 231, 241 against the microneedle structures 221, and driving the together through the tray 230, 240 and into the underlying skin.

### Microneedle Structures and Loading

With reference to FIG. 6, an exemplary microneedle drug delivery device comprises freestanding microneedle structures 221 supported by a deformable tray or elastomeric base layer 220. The microneedle structures 221 may be pre-formed or molded directly into the base layer 220. The microneedles 221 are designed to deliver their payload 231, 241 by penetrating through the deformable base layer 220 and into the underlying skin to an optimized depth for intracutaneous or transdermal administration.

In one embodiment, the microneedle structures 221 are integrally or superficially loaded with non-lyophilized therapeutic agents by known methods, as is standard practice known to those skilled in the art and practice of microneedle delivery manufacture and methods. Additionally or optionally, the microneedle structures 221 may be coated or wetted with additional active or inactive agents before application. In this way, the microneedle tips 221 may be loaded or coated with an active ingredient while the reservoir 230, 240 holds an auxiliary therapeutic payload, thereby supporting combination therapies and personalized dosing strategies. In a non-limiting example, a microneedle 221 intended to deliver an extruded implant may also include a thin hydrophilic coating, including but not limited to glycerin or polyethylene glycol, in order to assist in or enhance initial penetration as well as hydration and diffusion.

### Adhesive Tray Design

In an exemplary embodiment, the deformable tray 220 supporting the microneedles 221 may feature an adhesive layer on its lower surface, designed to adhere to the skin following application. In one aspect, the deformable tray 220 serves to anchor and stabilize the skin during delivery, thereby facilitating microneedle penetration. In another aspect, upon removal (e.g., by an application of force or pressure to move the device 200 away from the subject's skin) of one or more components of the device 200 relative to the subject's skin, the tray 220 may detach from the delivery device 220 and remain adhered to the subject's skin, thereby performing one or more secondary functions.

### Occlusion

In an exemplary embodiment, the adhesive deformable tray 220 may include a hydrogel layer or moisture barrier that maintains a hydrated environment for improved healing or enhanced diffusion of active agents, and/or limits contact with the external environment following microneedle penetration.

### Protective Barrier

In another exemplary embodiment, the adhesive tray 220 may provide a clean protective layer over the treated area to prevent environmental contamination or reduce irritation following microneedle penetration.

### Independent Transdermal Patch Functionality

In still another exemplary embodiment, the adhesive tray 220 may include an additional drug payload 231, 241 designed to exploit the transient increase in skin permeability following microneedle penetration. For example, analgesics, antibiotics, additional APIs, or cosmetic agents may be incorporated to deliver additional benefits. Additionally or optionally, the adhesive layer may further comprise materials such as: pressure-sensitive adhesives (PSAs) formulated for biocompatibility and extended wear; hydrocolloid materials with moisture-retentive properties; occlusive films or barrier coatings to minimize water loss and protect the treated area; penetration enhancers to promote uptake of secondary transdermal drug payloads; or ultraviolet absorbers to minimize photodegradation of payloads or photosensitization of skin following application. In one embodiment, the adhesive tray 220 is constructed to release from the microneedle delivery device automatically during removal, thereby ensuring smooth and consistent detachment without requiring manual intervention.

### Reservoir and Payload Design

In an exemplary embodiment, the microneedle device 200 further includes one or more reservoirs 230, 240 situated above the microneedle structures 221. These reservoirs 230, 240 may further contain delivery payloads 231, 241 each comprising: lyophilized vaccines or biologics; non-lyophilized solid or semi-solid therapeutic formulations; and/or combination therapies featuring immediate and/or sustained-release drug compositions.

In one embodiment, the reservoir channels 230, 240 may contain a dual- or multi-chamber system that allows more than one discrete payload 231, 241 to be simultaneously co-delivered. In one exemplary embodiment, a drug providing symptomatic relief, such as a pain-reliever, may be delivered from one chamber of the reservoirs 230, 240 while a second drug addressing the underlying disease state, such as an antibiotic, is delivered from a second chamber of the reservoirs 230, 240. Delivery of more than one active agent in a single administration provides a range of benefits, including but not limited to: reduction of the number of injections required, avoidance of storage incompatibilities between the actives due to physical separation in the respective reservoirs, reduced space requirements due to compact storage of multiple doses in a single device 200.

In another exemplary embodiment, the device 200 may deliver a primary payload that dissolves rapidly along with a secondary payload with a delayed or controlled-release characteristic. For example, an initial fast-acting pain reliever may be paired with a longer-acting anti-inflammatory agent. In another example, a rapidly dissolving primary vaccine dose may be co-delivered with a delayed onset secondary booster dose.

In one embodiment the reservoirs 230, 240 contents may be pre-selected and assembled into devices 200 to deliver pre-determined payload combinations. In another embodiment, specific payloads 231, 241 may be selected from a library of pre-loaded reservoirs at the time of use and loaded into the microneedle device 200 as appropriate to address a specific need or situation, including but not limited to an emergency response setting.

### Device Activation and Deployment

In an exemplary embodiment, the microneedle device includes a post assembly 210a designed to press the microneedles 221 through the deformable tray 220 and into the skin. In a non-limiting example, the post structures 210a may be configured to: compress the payload 231, 241 against the microneedle tips 221 before deployment; drive the microneedles 221 through the tray 220 and into the skin with controlled force; and/or in some embodiments to activate an adhesive tray release mechanism to ensure the adhesive layer remains on the skin when the device 200 (or components thereof) is withdrawn.

In one embodiment, the device 200 may include a spring-loaded actuator or trigger mechanism that ensures sufficient force is applied during deployment, thereby enhancing microneedle penetration depth and improving payload transfer efficiency. In one embodiment, the device 200 may comprise visual, haptic, auditory, or a combination thereof indicators to confirm deployment status and accomplishment of successful delivery.

In one embodiment, the device 200 may include one or more locking mechanisms to prevent accidental deployment, and/or to prevent accidental re-use. In one embodiment the locking mechanism may comprise a pull and/or breakaway tab, a locking pin, and/or a rotating feature.

In some embodiments, the device 200 may include guide or alignment structures 251 to ensure that posts 210a remain in alignment with, and deploy precisely through, the other features of the device 200, including reservoir channels 230, 240 and the deformable tray 220.

### Manufacturing and Filling

In some embodiments, the microneedle device 200 may be specifically designed for compatibility with existing automated aseptic filling lines, thereby simplifying integration into established or existing vaccine and drug manufacturing processes. The materials of construction may include ingredients that modify the thermal conductivity of the reservoirs or the entire device to facilitate lyophilization after liquid filling of the device. Such materials may include for example, boron nitride, aluminum oxide, aluminosilicates, graphite and graphene, or other ingredients. In some embodiments, the device 200 can be configured such that it may be handled and processed by equipment designed to process standard glass vials, without substantial modification to that equipment. In this form, a dose of vaccine or other therapeutic agent may be delivered through a top opening, and the internal configuration of the device may direct the liquid dose into reservoir features 230, 240 where it can be lyophilized in place as part of the pre-assembled device.

In some alternative configurations, the reservoir features 230, 240 can be loaded with materials independently, then subsequently assembled into a functional delivery device. In some embodiments, the device is designed to accept pre-formed implants or controlled-release materials inserted directly into the reservoir structure. A variety of methods may therefore be utilized to introduce a dose into each of the reservoirs in a reservoir plate. In some embodiments, different channels of the same reservoir plate 230, 240 may be loaded with different payloads 231, 241.

### Alternative Filling Method

In addition to compatibility with traditional aseptic filling lines, the inherent fixed volume of the reservoir channels 230, 240 enables alternative bulk-filling methods. By simply filling the reservoirs 230, 240 to capacity, the need for precise dispensing systems can be eliminated. For example, in one embodiment, one face of the reservoir channel plate 230, 240 can be brought into contact with an excess volume of a liquid, which is then drawn into the channels, filling them to capacity by capillary action. This approach simplifies manufacturing steps by leveraging the inherent volume constraints of the reservoir channels to ensure accurate dosing without precise liquid handling.

In some embodiments, the channel plates 230, 240 may be separately filled by capillary action, controlled surface tension effects, or immersion-based techniques that utilize the fixed volume of each channel to ensure a complete and precise dose is loaded. In other embodiments a solid or semisolid formulation may be loaded into channels 230, 240 by compression. These alternative method provides a robust and efficient solution for large-scale production of microneedle delivery devices 200, particularly for non-lyophilized materials or implant formulations that are not easily metered by traditional dispensing equipment.

In one embodiment, the reservoir system is configured to facilitate capillary filling of non-lyophilized liquid formulations. Features such as vent channels, hydrophobic coatings, or directed flow guides may be included to improve efficient loading of solid or semi-solid materials. In some embodiments the reservoir channels 230, 240 may be filled with dry powders or pastes, non-aqueous suspensions, or semi-solid formulations. Additionally or optionally, the reservoir channels 230, 240 may be loaded with pre-formed tablets or solid dosage forms, including bio-erodible implants and sustained release formulations.

### Clinical Applications and Use Cases

The present invention is further intended to expand the utility of microneedle devices 200 beyond conventional vaccine administration. Non-limiting examples of applications and use cases include: delivery of hormonal implants for contraception or hormone replacement therapy; administration of rescue medications such as receptor antagonists, sedatives, anti-venoms, and/or antidotes; prophylactic administration of protective agents prior to anticipated hazard exposure; and/or co-administration of multiple therapies for combination treatments. The expanded capabilities of the present invention therefore offer enhanced versatility for both developed and resource-limited healthcare settings, improving delivery options for a wider range of therapeutic agents.

Certain advantages of drug delivery device 200 include improved skin penetration characteristics by combining friction-enhancing engagement between the deformable tray 220 and the skin and controlled-force deployment via a post-actuated structure 210a that drives microneedles 221 past the lower surface of the tray 220 with enhanced penetration depth. Additionally, the expanded versatility of the present invention enables improved delivery of a wide range of therapeutic agents, including but not limited to: vaccines; biologics; peptides and proteins; hormone therapies; virus-like particles; nucleic acid-based therapies (e.g., mRNA, siRNA); analgesics and anti-inflammatory agents; regenerative medicine agents and growth factors; wound care and dermatological treatments. Still further, by integrating features such as adhesive trays, non-lyophilized payload compatibility, and improved skin engagement, the present invention represents a significant advancement in microneedle drug delivery, thereby offering a robust platform for improved therapeutic outcomes, greater manufacturing efficiency, and enhanced versatility across diverse clinical applications.

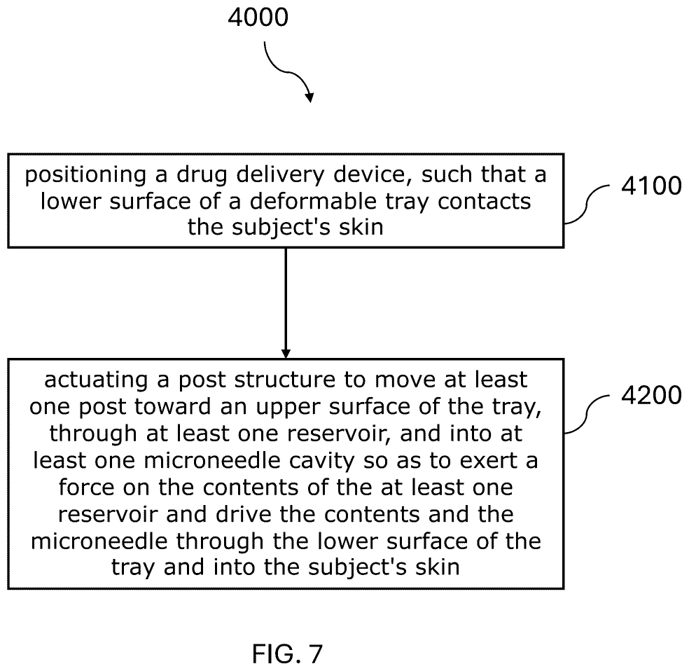

Referring now to FIG. 7, method 4000 of administering a drug to a subject is provided. The method includes step 4100 of positioning a drug delivery device, such as drug deliver device 200 described above, such that a lower surface of the deformable tray contacts the subject's skin. The method also includes step 4200 of actuating at least one post 210a toward an upper surface of the tray 220, through the at least one reservoir 230, 240, and into at least one microneedle 221 cavity so as to exert a force on the contents of the at least one reservoir 230, 240 and drive the contents and the microneedle 221 through the lower surface of the tray 220 and into the subject's skin. In an exemplary embodiment, the adhesive surface of the deformable tray 220 remains adhered to the skin upon removal of the microneedle device 200. Additionally or optionally, the post structure 210a is configured to deploy the microneedles 221 at a controlled force sufficient to drive the microneedles 221 to a depth of at least 0.5 mm, 0.8 mm, or 1 mm below the skin surface.

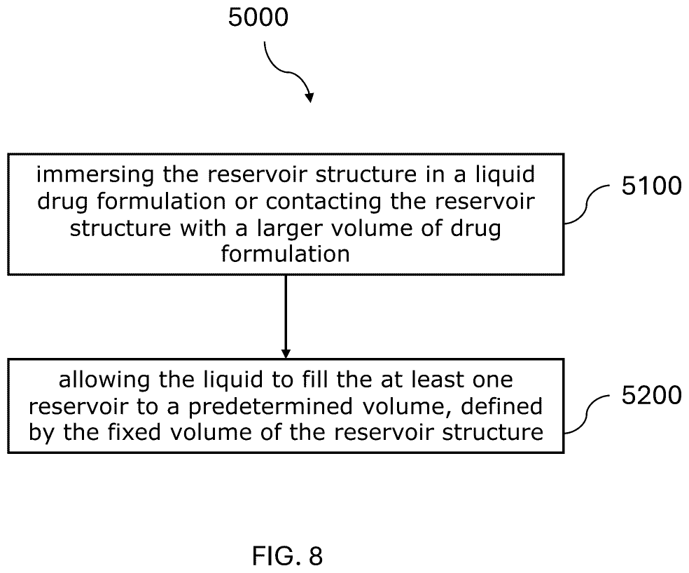

Turning now to FIG. 8, a method 5000 of filling a drug delivery device is provided. In an exemplary embodiment, the drug delivery device, such as device 200, comprising a post structure 210a, a deformable tray 220 defining at least one microneedle 221 cavity, and at least one reservoir 230, 240 axially aligned with the at least one microneedle 221 cavity. The method 5000 includes step S100 of immersing the reservoir structure 230, 240 in a liquid drug formulation or contacting the reservoir structure 230, 240 with a larger volume of drug formulation. The method 5000 also includes step S200 of allowing the liquid to fill the at least one reservoir 230, 240 to a predetermined volume as defined by the fixed volume of the reservoir structure 230, 240. In an exemplary embodiment, the reservoir structure 230, 240 includes vent channels or capillary action features that promote complete filling without precise metering. In still another exemplary embodiment, the method 5000 includes a step of freeze-drying the contents of the reservoir 230, 240 (after the filling step S200) to produce a stable lyophilized formulation. In yet another exemplary embodiment, the method 5000 includes a step of coating the microneedle tips 221 with a distinct therapeutic agent prior to filling the reservoir structure 230, 240.

Although the invention is illustrated and described herein with reference to specific embodiments, the invention is not intended to be limited to the details shown. Rather, various modifications may be made in the details within the scope and range of equivalents of the claims and without departing from the invention

What is claimed is:

1. A drug delivery device comprising:
a post structure comprising a base and at least one post protruding from the base;
a deformable tray movably coupled to the post structure, the deformable tray defining at least one cavity extending from an upper surface of the tray toward a lower surface of the tray, the at least one cavity tapering to a point at or near the lower surface of the tray;
at least one microneedle positioned within the at least one cavity;
at least one reservoir positioned directly above and axially aligned with the at least one cavity, the reservoir configured to contain a therapeutic agent; and
an adhesive surface positioned on the lower side of the deformable tray, the adhesive surface configured to adhere to the skin and optionally remain on the skin when the microneedle device is withdrawn.

2. The drug delivery device of claim 1, wherein the adhesive surface comprises a hydrogel layer configured to promote localized hydration, improve wound healing, or enhance transdermal diffusion.

3. The drug delivery device of claim 1, wherein the adhesive surface comprises a protective barrier configured to minimize exposure to environmental contaminants and reduce irritation following microneedle penetration.

4. The drug delivery device of claim 1, wherein the adhesive surface comprises an active agent designed for transdermal delivery following microneedle penetration, selected from analgesics, anti-inflammatoires, antibiotics, growth factors, or cosmetic agents.

5. The drug delivery device of claim 1, wherein the at least one reservoir comprises a dual-chamber system configured to deliver an immediate-release therapeutic payload and a sustained-release therapeutic payload.

6. The drug delivery device of claim 1, wherein the at least one reservoir is configured to receive a payload via bulk filling by immersion or by contacting the reservoir structure with a larger volume of drug product, wherein the fixed volume of the reservoir channel determines the filled dose.

7. The drug delivery device of claim 1, wherein the at least one reservoir comprises vent channels or capillary action features configured to facilitate passive filling during immersion or bulk contact with a liquid drug formulation.

8. The drug delivery device of claim 1, wherein the post structure is configured to press the at least one microneedle through the deformable tray and into the skin upon deployment.

9. The drug delivery device of claim 1, wherein the post structure is configured to compress the contents of the reservoir against the microneedle tips before deployment to ensure payload transfer during application.

10. The drug delivery device of claim 1, further comprising a spring-loaded actuator configured to ensure a minimum force threshold is achieved before microneedle deployment.

11. The drug delivery device of claim 1, wherein the at least one microneedle is pre-coated with a therapeutic agent selected from vaccines, biologics, peptides, proteins, or nucleic acid-based therapies.

12. A method of administering a drug to a subject, the method comprising:
positioning the drug delivery device of claim 1 such that the lower surface of the deformable tray contacts the subject's skin; and
actuating the post structure to move at least one post toward the upper surface of the tray, through the at least one reservoir, and into the at least one microneedle cavity so as to exert a force on the contents of the at least one reservoir and drive the contents and the microneedle through the lower surface of the tray and into the subject's skin.

13. The method of claim 12, wherein the adhesive surface of the deformable tray remains adhered to the skin upon removal of the microneedle device.

14. The method of claim 12, wherein the post structure is configured to deploy the microneedles at a controlled force sufficient to drive the microneedles to a depth of at least 1 mm below the skin surface.

15. A method of filling a drug delivery device, the device comprising a post structure, a deformable tray defining at least one microneedle cavity, and at least one reservoir axially aligned with the at least one microneedle cavity, the method comprising:
immersing the reservoir structure in a liquid drug formulation or contacting the reservoir structure with a volume of drug formulation; and
allowing the liquid to fill the at least one reservoir to a predetermined volume as defined by the fixed volume of the reservoir structure.

16. The method of claim 15, wherein the reservoir structure includes vent channels or capillary action features that promote complete filling without precise metering.

17. The method of claim 15, further comprising a step of freeze-drying the contents of the reservoir after the filling step to produce a stable lyophilized formulation.

18. The method of claim 15, further comprising a step of coating the microneedle tips with a distinct therapeutic agent prior to filling the reservoir structure.
